(19) United States
(12) Patent Application Publication
(10) Pub. No.: US 2024/0358421 A1
(43) Pub. Date: Oct. 31, 2024

(54) Resorptive Intramedullary Implant Between Two Bones or Two Bone Fragments

(71) Applicant: Stryker European Operations Holdings LLC, Kalamazoo, MI (US)

(72) Inventors: Bernard Prandi, Rennes (FR); Marc Augoyard, Tassin La Demi Lune (FR); Thomas Ledermann, Eschenbach (CH); Tristan Meusnier, Saint-Etienne (FR); Jacques Peyrot, Tassin La Demi Lune (FR); Judith Fellmann, Stafa (CH)

(21) Appl. No.: 18/770,767

(22) Filed: Jul. 12, 2024

Related U.S. Application Data
(63) Continuation of application No. 16/506,353, filed on Jul. 9, 2019, now Pat. No. 12,059,186.
Application No. 16/506,353 is a continuation of application No. 14/858,855, filed on Sep. 18, 2015, now Pat. No. 10,383,671.
Application No. 14/858,855 is a division of application No. 13/795,946, filed on Mar. 12, 2013, now Pat. No. 9,168,074.
Application No. 13/795,946 is a continuation of application No. 12/918,105, filed on Oct. 29, 2010, now Pat. No. 8,414,583.

(30) Foreign Application Priority Data
Sep. 9, 2008 (FR) 0856035

(51) Int. Cl. A61B 17/86; A61B 17/00; A61B 17/16; A61B 17/68; A61B 17/72; A61B 17/88; A61F 2/30; A61F 2/42

(52) U.S. Cl. CPC A61B 17/8605; A61B 17/1655; A61B 17/68; A61B 17/7233; A61B 17/88; A61F 2/4225; A61F 2/4241; A61B 2017/00862; A61B 2017/681; A61B 17/7208; A61B 17/7225; A61B 17/7266; A61B 17/7283; A61B 2017/8655; A61F 2002/30062; A61F 2002/30563; A61F 2002/30622; A61F 2002/3085; A61F 2002/30878; A61F 2210/0004

(57) ABSTRACT

The invention relates to a resorptive intramedullary implant between two bones or two bone fragments. The implant includes a single-piece body (1) having a generally elongate shape and having, at each end, areas for anchoring to the bone portions in question, characterized in that one of said areas (A1) has a cylindrical cross-section while the other area (A2) has a flat cross-section.

### CROSS-REFERENCE TO RELATED APPLICATIONS

This application is a continuation of U.S. patent application Ser. No. 16/506,353, filed on Jul. 9, 2019, which is a continuation of U.S. patent application Ser. No. 14/858,855, filed Sep. 18, 2015, now U.S. Pat. No. 10,383,671, which is a divisional of U.S. patent application Ser. No. 13/795,946, filed Mar. 12, 2013, now U.S. Pat. No. 9,168,074, which is a continuation of U.S. patent application Ser. No. 12/918,105, filed Oct. 29, 2010, now U.S. Pat. No. 8,414,583, which application is a U.S. national phase entry under 35 U.S.C. § 371 of International Application No. PCT/FR2009/051658, filed Sep. 2, 2009, published as WO 2010/029246, which claims priority from French Patent Application No. 0856035, filed Sep. 9, 2008, whose entire disclosures are herewith incorporated by reference.

### FIELD OF THE INVENTION

The invention relates to the technical field of orthopedic implants, particularly for arthrodesis and osteosynthesis.

More particularly, the invention relates to an intramedullary implant for arthrodesis between two bone parts or osteosynthesis between two bone fragments, particularly in the case of the hand or foot.

### BACKGROUND OF THE INVENTION

Different solutions have been proposed to achieve these functions.

For example, a solution comes from the teaching of patent application FR 2,884,406 [US 2008/0177262], of which the applicant of the present application is also the applicant. This patent describes an intramedullary osteosynthesis device constituted of an elongated body whose ends constitute anchor zones cooperating with the bone parts to be immobilized. The anchor zones are shaped and made of a material selected to enable insertion into the bone parts, then to ensure an anchor in the bone parts by preventing any rotational movement by resisting traction and by maintaining a compression force.

Another solution also comes from patent application FR 07.02003 [US 2010/0131014], also from the same applicant. This document describes an implant in the form of two anchor zones connected by a central zone and whose general shape is substantially inscribed in a very elongated rectangle of X-shape, so as to form in the anchor zones two legs adapted to move apart by elastic or shape-memory effect.

From this design, different criteria have been established to make the implant easy to place and efficient in order to create a primary and secondary stability for the osteosynthesis or arthrodesis site.

However, these solutions are not adapted for the case of an implant made of resorptive material.

### BRIEF SUMMARY OF THE INVENTION

From this state of the art, the object that the invention proposes to attain is further improving the anchor and the stability of the implant as well as its adaptation to the morphology of the implantation site when the implant is made of resorptive material.

To solve such a problem, a resorptive intramedullary implant between two bones or two bone fragments has been designed and developed; it is constituted, in a known manner, of a single-piece body having a general elongated shape with, at each end, zones for anchoring to the bone parts being considered. According to the invention, one of the zones has a cylindrical shape, whereas the other zone is flat.

Advantageously, the implant is made of a resorptive material whose mechanical properties are determined to last the time necessary for the consolidation, so that the implant is resorbed after six months. For example, the implant is composed of lactic acid polymer or copolymer (PLA, PGA . . . ).

Considering the specific mechanical characteristics of resorptive materials, and to solve the given problem of improving anchor and stability, the cylindrical cross-section is threaded and tapers in the direction of its free end.

To solve the given problem of enabling a deformation by elasticity, thus causing an expansion adapted to the geometry of the site and to the properties of the material, the flat cross-section zone has, substantially in its median portion, an opening adapted to enable elastic deformation of the zone. The opening defines at least two anchor arms.

It therefore appears that the combination of a cylindrical and threaded anchor zone and a flat-sectioned anchor zone is particularly advantageous considering the problem to be solved.

To solve the given problem of resisting the shear and flexion forces susceptible of occurring in the area of the bone site, between the two anchor zones, the body has a central zone of transition adapted to resist the shear and flexion forces occurring in the area of the bone site and adapted to serve as an abutment.

From this basic design of the implant, the anchor zones are either coaxial or angularly offset by between about 1° and 30° and, advantageously, by 10°. The bend between the anchor zones is located so as to substantially correspond to an arthrodesis line of the bones being considered.

### BRIEF DESCRIPTION OF THE DRAWINGS

The invention is explained in more detail hereinafter with reference to the attached drawings, in which:

FIG. 1 is a perspective view of the implant;

FIG. 2 is a front view of the implant before insertion into the bone part in question;

FIG. 3 is a side view corresponding to FIG. 2;

FIG. 4 is a view like FIG. 2 showing the position of the anchor arms of the flat section after insertion;

FIG. 5 is a perspective view of another advantageous embodiment of the implant;

FIGS. 6 and 7 show the installation of the implant into two bone parts.

### DETAILED DESCRIPTION

The implant according to the invention has a one-piece body 1 of elongated shape and having a first proximal zone A1 and a second distal zone A2. The entire implant body is made of a resorptive material whose mechanical properties are determined for the implant to be resorbed in no less than about 6 months. In one embodiment, the implant is composed of lactic acid polymer or copolymer (PLA, PGA . . . ).

As will be described later in the description, the zones A1 and A2 have anchor formations for the respective bone parts. Taking into account the specific characteristics of the resorptive material and to attain the given object of anchor and stability, the zone A1 is of a cylindrical shape section whereas the other zone A2 is flat.

The zone A1 has a generally cylindrical outer surface 1a with a limited taper toward its free end. The surface 1a has a helical rib forming a screwthread 1a1.

The zone A2 is flat and has substantially in its center, an opening 1b adapted to enable elastic deformation of the zone A2. More particularly, the opening 1b defines at least two anchor arms 1c and 1d, each having at least one outwardly projecting tooth 1c1, 1d1.

Advantageously, between the two zones A1 and A2 the body 1 has a central zone C for transition adapted to resist shear and flexion forces that can occur at the end of a bone. By way of nonlimiting example, this median zone C can have a length of about 3.5 mm and a thickness of about 2 mm, for an overall implant length comprised between about 15 and 25 mm and a diameter of about 2 or 3 mm at the zone A1.

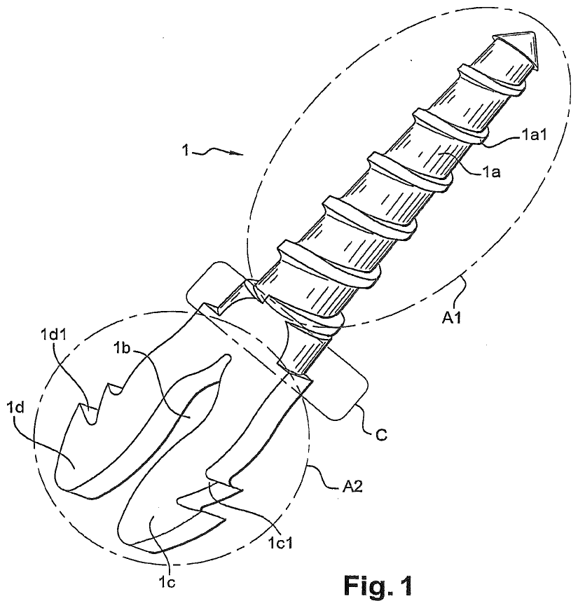

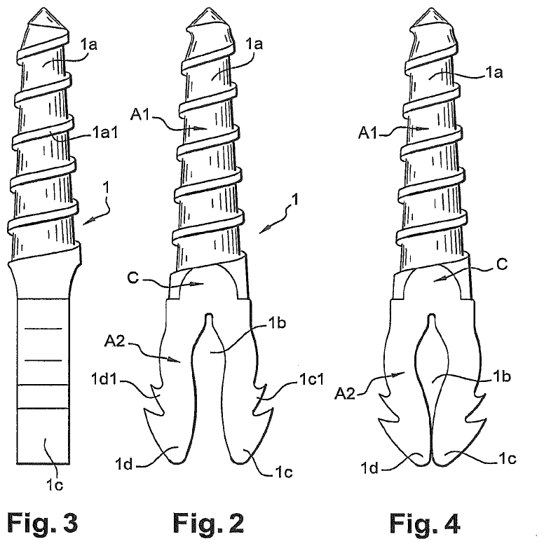

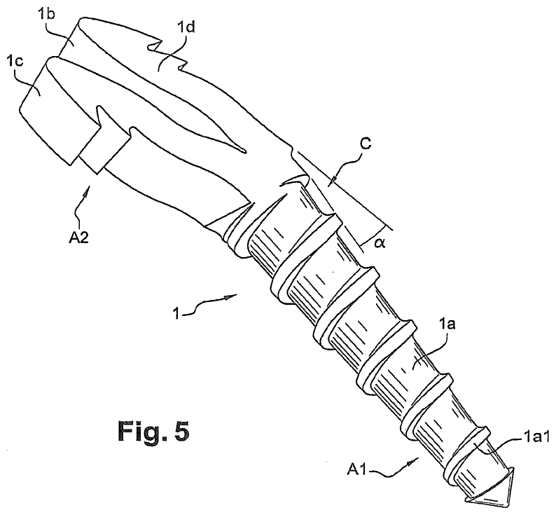

In the embodiment shown in FIG. 1, the two zones A1 and A2 are coaxial.

To solve the problem of adaptation to the shape of the implantation site, the anchor zones A1 and A2 can be offset at an angle α adapted to the geometry of the bone site. This angle α is comprised between about 1° and 30° and, advantageously, on the order of 10° when the implant is for foot arthrodesis (FIG. 5).

In this embodiment in which the two anchor zones are angularly offset, the bend is located so as to correspond substantially to the arthrodesis line of the bone parts being fused.

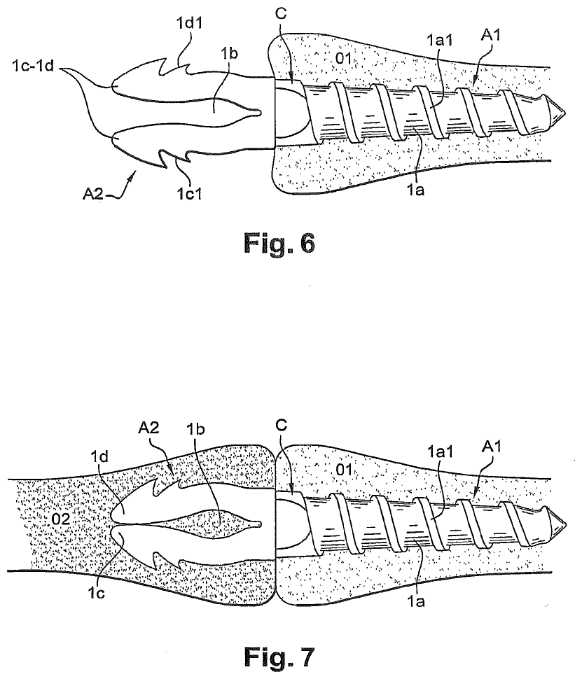

FIGS. 6 and 7 schematically show the positioning of the implant according to the invention between two bone parts O1 and O2. After suitable holes have been made in the bone by a rasp-type tool, the operator screws the thread 1a into the bone part O1 substantially up to the median zone C that serves as abutment preventing the implant from sinking too deeply into the bone (FIG. 6). The operator then fits the second bone part O2 back onto the anchor arms 1d and 1c of the zone A2, the anchor arms then spread and tighten by elasticity (FIG. 7).

The operative technique can be the following:
Drilling of the two holes with a conventional drill;
Preparation of the holes with a rasp for the flat side and a bone tap to form the inner screw thread on the cylindrical side;
Use of a screwdriver with a gripper end;
Screwing in the cylindrical side P1 [A1] for an arthrodesis IPP of the foot;
Fitting of the bone back onto the flat side [A2] of the implant.

The advantages are readily apparent from the description; in particular, it is to be emphasized and understood that the combination of the two anchor zones A1 and A2 of cylindrical and a flat shape, respectively, significantly enhances anchor and stability of the implant adapted to the geometry of the bone site and to the material properties, namely, a resorptive material.

1. (canceled)

2. A method for fixing first and second bone parts with respect to each other, the method comprising the steps of:
threading a first end of an implant into the first bone part; and
fitting first, second and third teeth of a second end of the implant into the second bone part such that the first tooth contacts a first side of the second bone part at a first portion, the second tooth contacts the first side at a second portion of the second bone part, and the third tooth contacts a second side of the second bone part opposite the first side, the first and second portions being spaced apart from one another.

3. The method of claim 2, wherein the threading step includes threading a tapered end of the first end into the first bone part, the tapered end tapering in a direction away from the second end.

4. The method of claim 2, wherein the fitting step includes elastically deforming the second end.

5. The method of claim 4, wherein the elastically deforming step include moving two spreadable arms separated by an opening with respect to each other.

6. The method of claim 2, further comprising the step of preventing overinsertion of the implant into the second bone part.

7. The method of claim 6, wherein the preventing step includes contacting an abutment with the second bone part.

8. The method of claim 7, wherein a face of the abutment defines a plane perpendicular to the longitudinal axis of the first end.

9. The method of claim 2, wherein a longitudinal axis through the first end is offset from the longitudinal axis of the second end by an angle less than 30 degrees.

10. The method of claim 9, further comprising the step of aligning the offset at a position corresponding substantially to an arthrodesis line defined at the intersection of the first and second bone parts.

11. The method of claim 2, further comprising the step of permitting the implant to resorb after implantation.

12. The method of claim 2, wherein the fitting step includes positioning the first and third teeth at the same axial location in the second bone part.

13. The method of claim 2, further comprising the step of contacting opposing flat surfaces of the second end with the second bone part.

14. The method of claim 2, wherein the fitting step includes contacting a first flat surface of the first tooth and a second flat surface of the second tooth with the second bone part, the first and second flat surfaces being coplanar.

15. The method of claim 2, wherein the fitting step includes fitting a non-circular cross-section of the second end with the second bone part.

16. The method of claim 2, wherein the fitting step includes contacting a first flat surface of the first tooth, a second flat surface of the second tooth, and a third flat surface of the third tooth with the second bone part, the first, second, and third flat surfaces defining planes parallel to each other.

17. A method for implanting an intramedullary implant between first and second bone parts, the method comprising the steps of:
threading a first end of the implant into the first bone part; and
fitting first, second and third teeth of a second end of the implant into the second bone part such that the first tooth contacts a first side of the second bone part at a first portion, the second tooth contacts the first side at a second portion of the second bone part, and the third tooth contacts a second side of the second bone part opposite the first side, the first and second portions being spaced apart from one another, wherein the fitting step includes elastically moving two spreadable arms of the second end with respect to each other.

18. The method of claim 17, wherein the two spreadable arms separated by an opening.

19. The method of claim 17, further comprising the step of preventing overinsertion of the implant into the second bone part by contacting an abutment with the second bone part.

20. The method of claim 17, wherein the fitting step includes contacting a first flat surface of the first tooth and a second flat surface of the second tooth with the second bone part, the first and second flat surfaces being coplanar.

21. The method of claim 17, wherein the fitting step includes contacting a first flat surface of the first tooth, a second flat surface of the second tooth, and a third flat surface of the third tooth with the second bone part, the first, second, and third flat surfaces defining planes parallel to each other.
